(19) United States
(12) Patent Application Publication
(10) Pub. No.: US 2011/0046414 A1
(43) Pub. Date: Feb. 24, 2011

(54) Photo-reaction of tyrosine with cetyltrimethylamonium chloride and nitrogen containing functional groups

(76) Inventor: Jian Zhang, Pomona, NY (US)

Correspondence Address: Jian Zhang, #174, 1581 Route 202, Pomona, NY, 10970-2901, US

(21) Appl. No.: 12/583,454

(22) Filed: Aug. 21, 2009

(51) Int. Cl. C07C 229/04

(52) U.S. Cl. 562/553

(57) ABSTRACT

A photo-reaction of tyrosine (and small tyrosyl peptides) with cetyltrimethylamonium chloride in aqueous buffer solution is described in this patent. The reactants were exposed to simulated sun irradiance of 760 W/m² at 30° C.±1° C. A reddish pigment was formed with the yield ranging from 60-100% in 6 hours depending on the peptides. The pigment was believed to be a melanin crossed linked with cetyltrimethylamonium chloride. Interaction between tyrosine and neighboring nitrogen containing groups was also observed. This reaction is useful for the research of some cosmetic and pharmaceutical products. It may also have other applications if other phenols and amines are used as reactants. Some advantages of this reaction include the mild reaction condition, simple procedures, and no need for oxidants, catalysts, organic solvents and heating.

### BACKGROUND

Tyrosine is one of the common amino acids in skin proteins. It has been reported that the photo-oxidation of tyrosine results in forming a pigment called melanin that leads to skin tanning, and that overexposure to sun can damage the skin tissues. ^{1,2} Some household products and cosmetics contain quaternary ammonium salt surfactants as antiseptics or conditioning agents. It is interesting to investigate any chemical interaction between these surfactants and labile amino acids if the products are intended to stay on the skin and would be exposed to sun lights.

The photo-stability of aromatic amino acids in protein drugs is also of considerable interest to pharmaceutical manufacturers.³ Despite that cationic surfactants are seldom used in products for internal use, amino acids such as tyrosine can interact with neighboring charged residues on the same protein molecule. Hence, a good understand of the interaction between amino acids will aid formulation scientists in developing stable protein products and packages.

According to literature, the initial product of tyrosine photo-oxidation (often by ionizing radiation) is dihydroxylphenylalanin (Dopa) which will turn into quinone and melanin subsequently.^{1,2,4} The structure of melanin is not very well defined and is thought to be represented by formula (I) in Scheme 2 and has the molecular weight ranging from 500 to 30,000 Da.^{1,2,4} Some experiments were carried out previously to react phenols (or quinones) and amines in the presence of oxidizing agents, catalysts, organic solvents, reactive starting materials and/or with heating.^{5,6,7} The products of these reactions were quinone imines or amino substituted phenols. This study reports a reaction of tyrosine and tyrosyl peptides (Scheme 1) with cetyltrimethylamonium chloride in aqueous buffer solution that is induced by sun light. Comparison of the cationic surfactant with anionic and nonionic surfactants in the photo-reaction is also made.

### DETAILED DESCRIPTION OF THE INVENTION

### Methods

The tyrosyl compounds were dissolved in 0.01 M ammonia acetate solution at a concentration of 2 mM. The pH of solutions was adjusted to 10. To the peptide solutions, 0.5% (wt/wt) sodium lauryl sulfate (anionic), cetyltrimethylammonia chloride (cationic), and polysorbate 80 (nonionic) were added. The solutions were exposed to simulated solar radiation (290-800 nm) in a Xenon Lamp chamber. The irradiance to which samples were exposed was set at 760 W/m². The temperature inside the chamber was maintained at 30° C.±1° C. by a cooler. Aliquots of samples were taken from the reacting solutions at 5 different time points within 6 hours and analyzed by a HPLC/UV/MS system. All the studies were conducted in duplicate.

### Results and Discussion

FIG. 1 shows that cetyltrimethylammonium chloride significantly increased the rate of photo-oxidation of the tyrosyl compounds compared to sodium lauryl sulfate and polysorbate 80. This suggests that a chemical interaction took place between cetyltrimethylammonium chloride and tyrosine. The reaction proceeded to 100% completion for Tyr-Arg and to 60% completion for Ac-Tyr in 6 hours as the fastest and slowest among all the compounds. In addition, tyrosine, Gly-Tyr, Glu-Tyr, and N-Ac-Tyr showed larger increase in reaction rates than Tyr-Arg and Lys-Tyr-Lys due to the electrostatic and steric effect for the latter. It is also possible that the guanidine and amino groups of Arg and Lys residues reacted with tyrosine through the similar mechanism as cetyltrimethylammonium chloride. The oxidation of tyrosine and Lys-Tyr-Lys was slightly sensitized by Tween 80, which may be attributed to the interaction of the amino groups on the Lys residues with the Tween 80 when they were activated by light.

The reaction product was a complex mixture of multiple products which eluted from reversed phase chromatography as a broad solvent front peak. It has a reddish color. The λₘₐₓ of the UV spectrum of tyrosine shifted from 275 nm to the visible range as the reaction progressed. The product was well suspended in water because cetyltrimethylammonium chloride itself is a surfactant. FIG. 2 shows the representative mass spectra of the tyrosine (MW 181) and the reaction products. The spectra indicate that the monomeric and polymeric melanins were formed in this reaction. The single quadruple mass spectrometer used in this experiment is able to detect masses up to 700 Da. Higher masses may be observed by more sophisticated equipment. A species with a weight gain of 26 Da was detected with Tyr-Arg (MW 338) which is consistent with adding the mass of a —CN group. The simplified formulas of these products are showed in Scheme 2. It is not yet determined which atom of the phenolic group was the new bonding site. Tyr-Arg is the least stable among all the test tyrosyl compounds. It degrades even at neutral pH, at which the reaction rate of the other compounds is much lower in the absence of cetyltrimethylammonium chloride. The instability of Tyr-Arg may be attributed to the interaction between the tyrosine residue and the Arg side chain. Furthermore, many hydroxylated by-products including Dopa were also observed in the reaction mixture of tyrosine, Tyr-Arg, and Lys-Tyr-Lys. These by-products can be minimized by lowering light intensity or removing oxygen in the solution. The amino terminus of tyrosine residue in peptides does not cyclize with the aromatic ring as easily because it requires the cleavage of the peptide bond although this was detected to a very small extent in this reaction. This may explain the slower reaction rate and less polymeric by-products observed with Gly-Tyr, Glu-Tyr and Ac-Tyr than tyrosine. It is apparent that the extent of tyrosine oxidation is related to the presence of nitrogen containing groups nearby. In addition, cetyltrimethylammonium chloride appears to catalyze the hydrolysis of one of the peptides (Gly-Tyr) under this reaction condition.

This compatibility study was performed with a two-component system. The actual products usually contain multiple ingredients besides the ammonium salt at various concentrations. The pH of the products can also be different. It will be wise to conduct a multi-component compatibility test by mixing the ammonium salt with other ingredients as in a real product.

Another conceivable application of this reaction is to the pigment manufacture industry using less expensive phenols instead of tyrosine as the reactant. The products of this reaction can be used as a pigment material which is generated under a mild reaction condition without oxidizers, catalysts, organic solvents and heating involved. This method can also generate some useful intermediates for organic synthesis. For example, when catechol and resorcinol (both are dihydroxylbenzene) were mixed with ethanolamine and tetrabutylamonium salts and exposed to natural sun light, changes in the color and UV/Vis spectra of the reaction mixtures were observed as shown in FIG. 3. The reaction rate of catechol is faster than resorcinol because the two hydroxyl groups of catechol are in ortho-positions. HPLC analysis indicates that catechol was completely converted to the products.

### SUMMARY

A photo-reaction of tyrosine (and small tyrosyl peptides) with cetyltrimethylamonium chloride in aqueous buffer solution is described in this patent. The reactants were exposed to simulated sun irradiance of 760 W/m² at 30° C.±1° C. A reddish pigment was formed with the yield ranging from 60-100% in 6 hours depending on the peptides. The pigment was believed to be a melanin crossed linked with cetyltrimethylamonium chloride. Interaction between tyrosine and neighboring nitrogen containing groups was also observed. This reaction is useful for the research of some cosmetic and pharmaceutical products. It may also have other applications if other phenols and amines are used as reactants. Some advantages of this reaction include the mild reaction condition, simple procedures, and no need for oxidants, catalysts, organic solvents and heating.

### REFERENCES

1. Huggins T G, Wells-Knecht M C, Detorie N A, Baynes J W, Thorpe S R. Formation of o-tyrosine and dityrosine in protein during radiolytic and metal-catalyzed oxidation. J Biol Chem. 1993; 268: 12341-12347.

2. Joshi P, Carraro C, Pathak M. Involvement of reactive oxygen species in the oxidation of tyrosine and dopa to melanin and in skin tanning. Biochem Biophy Res Comm. 1987; 142: 265-274.

3. Kerwin B A Jr, Remmele R L Jr. Protect from light: photodegradation and protein biologics. J Pharm Sci. 2007; 96: 1468-1479.

4. Creed D. The photophysics and photochemistry of the near-UV absorbing amino acids-II. tyrosine and its simple derivatives. Photochemistry and Photobiology. 1984; 39(4): 563-575.

5. Hoch H and Scheuermann H. Diarylamines. German Patent No. 2544504. Apr. 14, 1977

6. Kallmayer H-J. Quinone-amine reactions, 1. Reactions between 2-methyl-1,4-naphthoquinone and 4-aminophenol. Arch. Pharm (German). 1979; 312(3): 230-239.

7. Hackman R H and Todd A R. Some Observations on the Reaction of Catechol Derivatives with Amines and Amino Acids in Presence of Oxidizing Agents. Oxidation of Catechol Derivatives, 1953; 55: 631-637.

### BRIEF DESCRIPTION OF THE DRAWINGS

SCH. 1: Structures of tyrosine, N-Ac-Tyr, Gly-Tyr, Glu-Tyr, Tyr-Arg and Lys-Tyr-Lys

SCH. 2: Proposed structures of melanin (I), crossed-linked melanin (II) and cyanotyrosine (III) as in Tyr-Arg

FIG. 1: Effect of surfactants on the rate of photo-oxidation of model tyrosyl compounds (2 mM) at pH 10 (irradiance=760 W/m²)

FIG. 2: Mass spectra of tyrosine (I), melanin (II), the monomer of melanin (III) and cyanotyroine in Tyr-Arg (IV)

FIG. 3: UV/Vis spectra of the reaction mixtures of catechol and two ammonium salts.

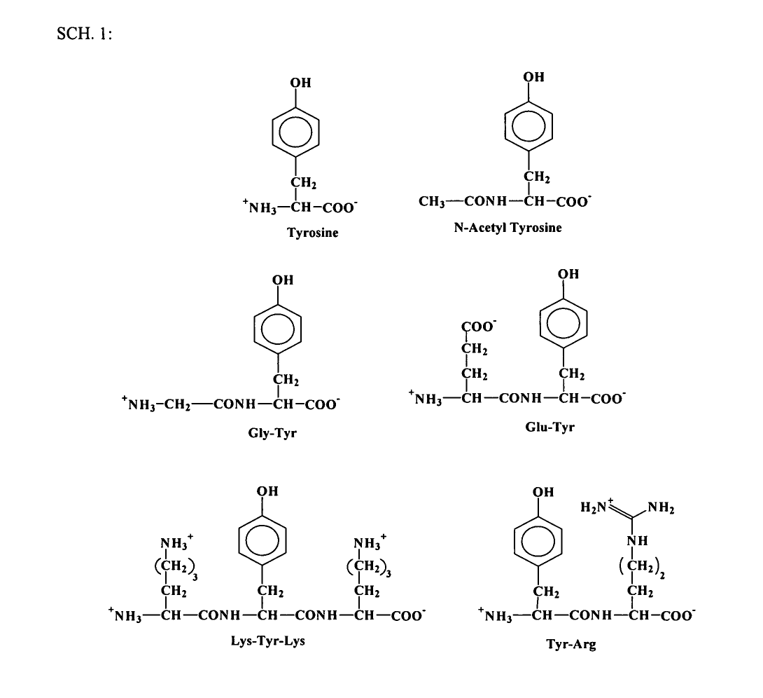

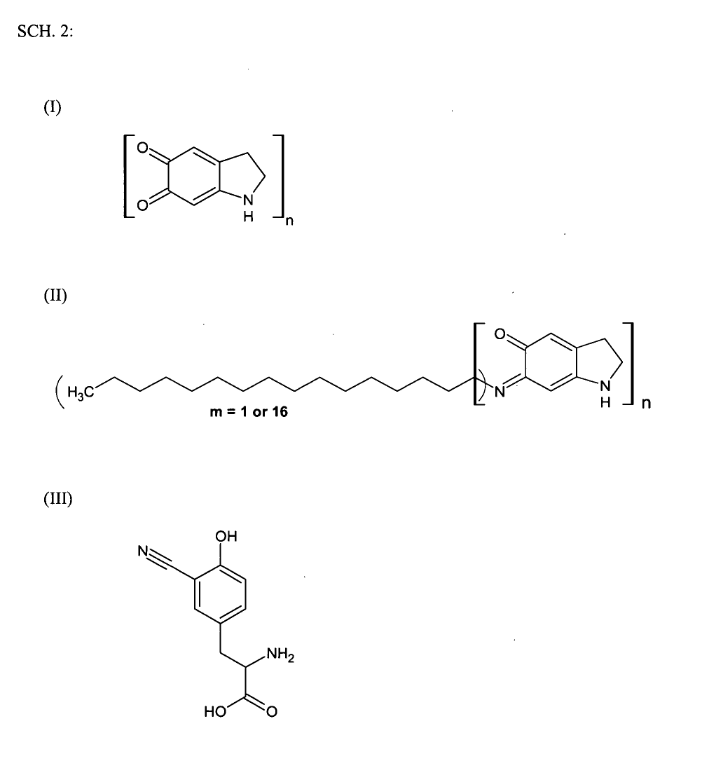

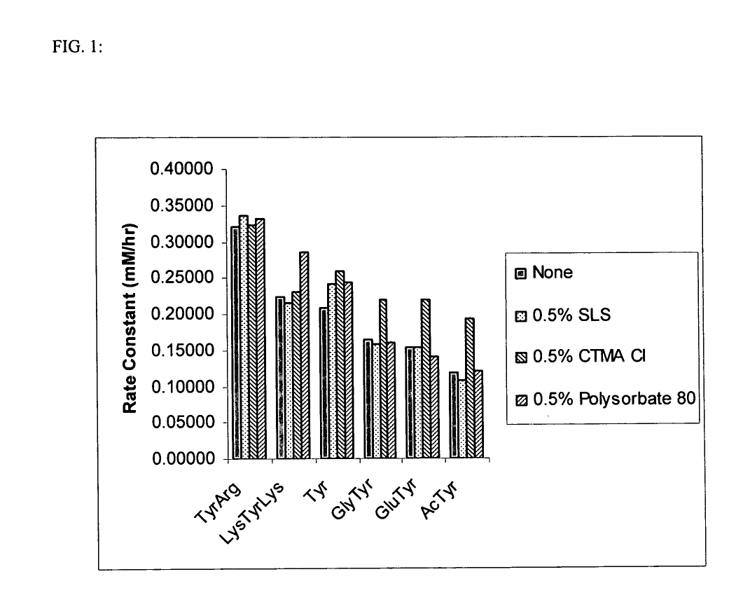

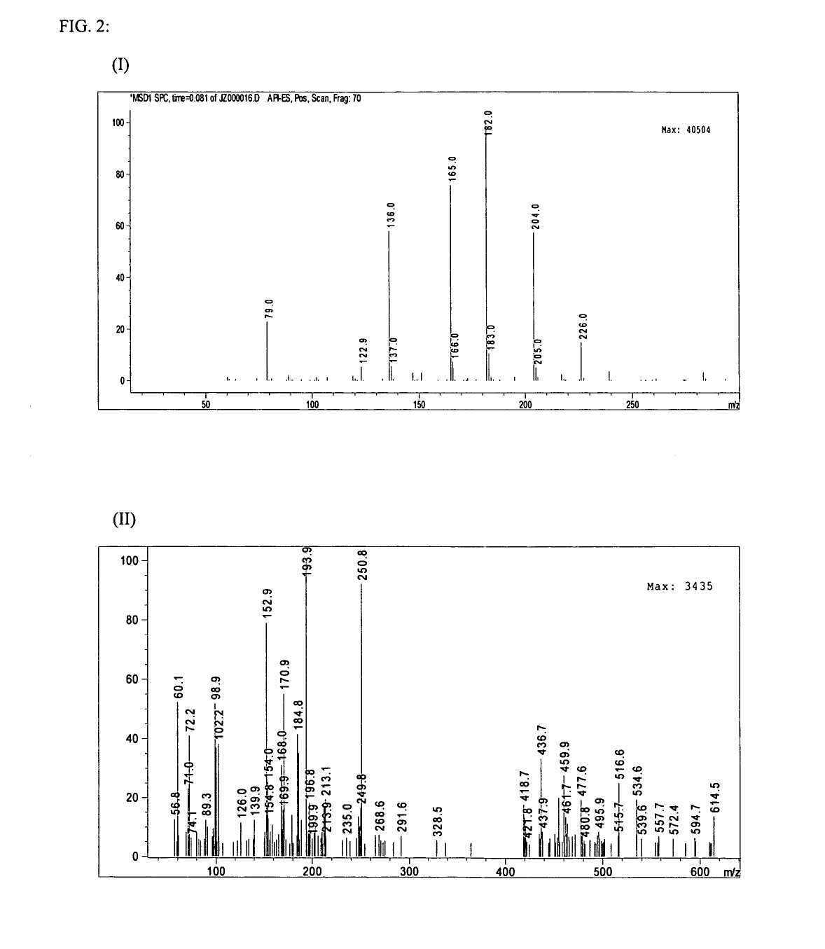

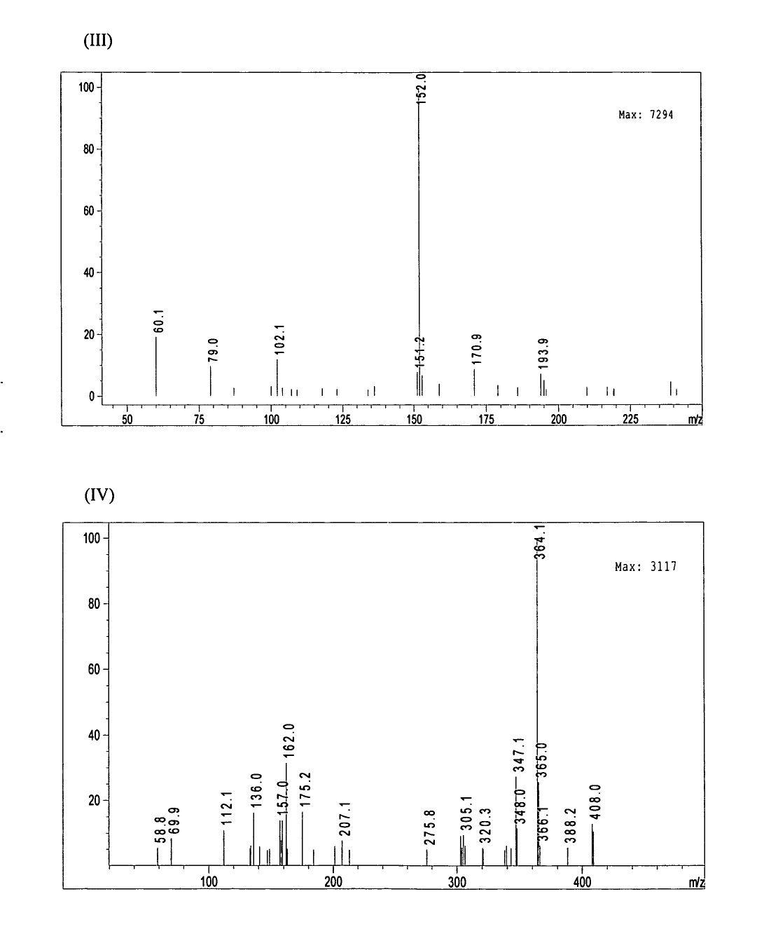

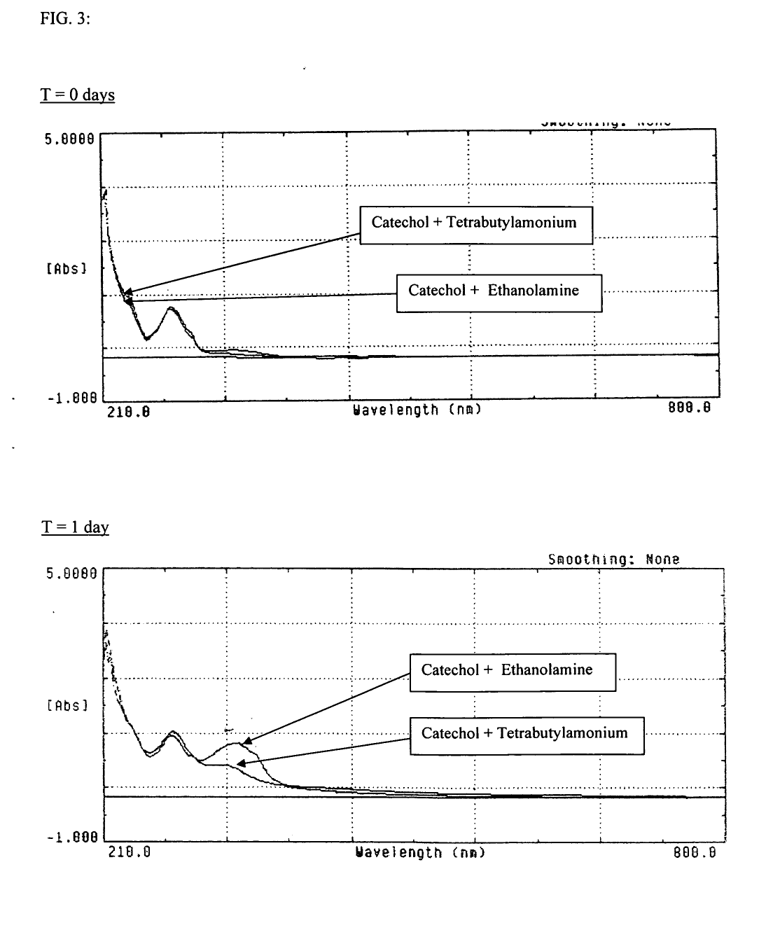

1. Tyrosine (and small tyrosyl peptides) reacts with cetyltrimethylamonium chloride in aqueous buffer solution when they are exposed to simulated sun light at 30° C. A reddish pigment is formed with the yield ranging from 60-100% in 6 hours depending on the peptides. The pigment was believed to be a melanin crossed linked with cetyltrimethylamonium chloride. Interaction between tyrosine and neighboring nitrogen containing groups was also observed.

2. This reaction is useful for the research of some cosmetic and pharmaceutical products. Other phenols and amines can also be used to react in the same manner with sun light. Hence this reaction may have other applications.
